(19) United States
(12) Patent Application Publication
(10) Pub. No.: US 2022/0202602 A1
(43) Pub. Date: Jun. 30, 2022

(54) METHODS OF USING REMOVABLE ORAL DEVICES

(71) Applicant: SCIENTIFIC INTAKE LIMITED CO., Lawrence, MA (US)

(72) Inventors: William H. Longley, Atlanta, GA (US); Richard P. Schneider, Bedford, NY (US); Anthony R. Tremaglio, Waban, MA (US); Marc M. Gibeley, Boxford, MA (US)

(21) Appl. No.: 17/495,528

(22) Filed: Oct. 6, 2021

Related U.S. Application Data
(63) Continuation of application No. 15/702,552, filed on Sep. 12, 2017, now abandoned.
(60) Provisional application No. 62/521,498, filed on Jun. 18, 2017.
(60) Provisional application No. 62/477,760, filed on Mar. 28, 2017.

(51) Int. Cl. A61F 5/00; A61B 5/00

(52) U.S. Cl. CPC A61F 5/0006; A61B 5/682; A61B 5/0537; A61B 5/4833; A61B 5/4866; A61B 5/4836

(57) ABSTRACT

Certain configurations of methods of using removable oral devices are described. In some examples, a removable oral device can be used in weight loss, weight management, athletic performance or in other applications to monitor or alter a user's behavior or monitor one or more physiological conditions. If desired, the removable oral device can be used in combination with a storage case, a mobile device, application software or other components.

### PRIORITY APPLICATIONS

This application claims priority to and the benefit of U.S. 62/477,760 filed on Mar. 28, 2017 and entitled “METHODS OF USING REMOVABLE ORAL DEVICES” and claims priority to U.S. Provisional Application No. 62/521,498 filed on Jun. 18, 2017 and entitled “METHODS OF USING REMOVABLE ORAL DEVICES.” The entire disclosure of each of these applications is hereby incorporated herein by reference for all purposes.

### CROSS-REFERENCE TO RELATED APPLICATIONS

This application is related to, and incorporates by reference herein, each of the following patent applications: U.S. Provisional Application 62/477,752 filed on Mar. 28, 2017 and entitled “REMOVABLE ORAL DEVICES,” U.S. Provisional Application 62/477,764 filed on Mar. 28, 2017 and entitled “METHODS OF PRODUCING REMOVABLE ORAL DEVICES,” U.S. Provisional Application 62/477,766 filed on Mar. 28, 2017 and entitled “SYSTEMS INCLUDING REMOVABLE ORAL DEVICES,” and U.S. Provisional Application 62/477,768 filed on Mar. 28, 2017 and entitled “REMOVABLE ORAL DEVICES AND THEIR USE IN COMBINATION WITH PHARMACOLOGICAL AGENTS, IMPLANTS AND OTHER DEVICES.”

### TECHNOLOGICAL FIELD

This application is directed to methods of using removable oral devices. More particularly, certain configurations described herein are directed to methods of using removable oral devices in weight management, athletic performance and other uses.

### BACKGROUND

Many methods for controlling weight exist. Most existing methods do not provide long term weight loss or health benefits.

### SUMMARY

Certain illustrative configurations are directed to various methods which use removable oral devices that can be inserted into the mouth and removed from the mouth, e.g. without the use of any tools, fasteners, insertion devices, etc. As noted in more detail below, the removable devices can be configured to reduce the overall usable volume of the mouth when solid food is received by the mouth to assist in weight management. In other instances, the removable oral device can be used to measure various physical parameters indicative of athletic performance, one or more physiological conditions or to monitor intake of pharmacological agents such as drugs. The methods described herein are generally non-surgical methods of weight loss or weight management, though if desired the methods described herein can be used in combination with surgical weight loss/control methods and/or one or more pharmacological agents designed to promote weight loss or control weight.

The methods described herein can use a removable oral device comprising a palatal element coupled to an optional clasping element. The palatal element is configured to contact the roof of a user's mouth at a palatal surface. The palatal element may comprise a body comprising a thickness configured to lower a vaulted area of the roof of the mouth to decrease an overall oral volume of the mouth to a first oral volume. The clasping element, when present, is configured to assist in removal of the removable oral device from the mouth. If desired, the palatal element may comprise a variable hardness, e.g., the edges of the palatal element can be softer than a central or apex surface of the palatal element.

In one aspect, a method of managing body weight in a human comprises providing a removable oral device, monitoring usage frequency and providing feedback to the human based on transferred usage values. For example, the method comprises providing a removable oral device comprising an integral usage sensor, wherein the removable oral device comprises a palatal element coupled to a clasping element, wherein the palatal element is configured to contact the roof of a user's mouth at a palatal surface, wherein the palatal element comprises a body comprising a thickness configured to lower a vaulted area of the roof of the mouth to decrease an overall oral volume of the mouth to a first oral volume, wherein the clasping element is configured to assist in removal of the removable oral device from the user's mouth. The method may also comprise monitoring usage frequency of the removable oral device by transferring usage values from the usage sensor to an external device separate from the removable oral device. The method may also comprise providing feedback to the human based on the transferred usage values to manage body weight in the human.

In certain embodiments, the method comprises transferring the usage values from the usage sensor to a removable oral device storage case sized and arranged to receive and store the removable oral device.

In other embodiments, the method comprises transferring the usage values from the stored removable oral device to the storage case by providing an optical transmission from the stored removable oral device to the storage case. In some examples, the method comprises the optical transmission to comprise non-visible light wavelengths, e.g., infrared light, ultraviolet light or other light outside of a wavelength range of 400-700 nm. If desired, however, visible light could also be used.

In some examples, the method comprises transferring the usage values received by the storage case to the external device.

In other examples, the method comprises transferring the usage values received by the storage case to a mobile device.

In some instances, the method comprises transferring the usage values received by the storage case to a wearable device.

In some configurations, the method comprises providing feedback to the human based on the transferred usage values to manage body weight in the human using a mobile device, e.g., one comprising application software.

In other configurations, the method comprises providing feedback to the human based on the transferred usage values to manage body weight in the human using a wearable device, e.g., one comprising application software.

In some examples, the method comprises providing feedback to the human based on the transferred usage values to manage body weight in the human using an in-person coach or a live coach, e.g., a human coach.

In some configurations, the removable oral device comprises a variable hardness with a hardness at an apex surface being greater than a hardness at edges of the removable oral device adjacent to teeth of the human when the removable oral device is inserted into the oral cavity of the human. For example, a Vickers hardness value (HV) at an apex surface is higher than a HV at the edges of the palatal element. In other examples, the method comprise configuring an apex surface of the removable oral device with a first material comprising a Vickers hardness of at least 10 HV and configuring edges of the removable oral device to comprise a second material with a Vickers hardness of less than 10 HV.

In certain examples, the method comprises configuring the usage sensor to comprise a temperature sensor, transferring the usage values from the usage sensor to a removable oral device storage case using a non-visible light transmission from the removable oral device to the removable oral device storage case, and wirelessly transferring the usage values received by the removable oral device storage case to a mobile device.

In some examples, the method comprises configuring the clasping element to comprise a wire comprising a coating on at least some portion of the wire, e.g., the coating may be similar or the same as one or more of the materials present in the apex surfaces or edges of the palatal element.

In certain configurations, the method comprises configuring the usage sensor to comprise one or more of an optical sensor, a Bluetooth device, a temperature sensor, a cellular chip, a radio frequency transmitter, a processor, a power supply, a battery, a memory unit, a transmitter or a receiver.

In another aspect, a method of managing body weight in a human by reducing the oral volume of the human during food intake comprises monitoring usage frequency of a removable oral device by transferring usage values from a usage sensor of the removable oral device to an external device separate from the removable oral device, wherein the removable oral device comprises a palatal element coupled to a clasping element, wherein the palatal element is configured to contact a roof of a user's mouth at a palatal surface, wherein the palatal element comprises a body comprising a thickness configured to lower a vaulted area of the roof of the mouth to decrease an overall oral volume of the mouth to a first oral volume, wherein the clasping element is configured to assist in removal of the removable oral device from the user's mouth, and wherein the palatal element comprises a variable hardness with a hardness at an apex surface being greater than a hardness at edges of the removable oral device adjacent to teeth of the human when the removable oral device is inserted into the oral cavity of the human. If desired, the method may also comprise providing feedback to the human based on the transferred usage values to manage body weight in the human.

In certain instances, the method comprises configuring the feedback to comprise one or more of an automated audio message, an automated video message or an automated text message in response to the transferred usage values.

In some examples, the method comprises transferring the usage values from the usage sensor to a removable oral device storage case sized and arranged to receive and store the removable oral device.

In other examples, the method comprises transferring the usage values from the stored removable oral device to the storage case by providing an optical transmission from the stored removable oral device to the storage case.

In some embodiments, the method comprises configuring the optical transmission to comprise non-visible light wavelengths.

In certain examples, the method comprises transferring the usage values received by the storage case to the external device.

In other embodiments, the method comprises transferring the usage values received by the storage case to a mobile device.

In certain instances, the method comprises transferring the usage values received by the storage case to a mobile device comprising application software, and providing feedback to the human based on the transferred usage values by remotely accessing the transferred usage values present in the application software.

In some examples, the method comprises configuring the provided feedback to alter the food intake of the human.

In other examples, the method comprises configuring the provided feedback to alter usage frequency of the removable oral device by the human.

In further examples, the method comprises configuring the provided feedback to alter an exercise schedule of the human.

In some examples, the method comprises configuring the provided feedback to alter a meal schedule of the human.

In other examples, the method comprises configuring the provided feedback to alter a meal frequency of the human.

In another aspect, a method of monitoring intake of material into a mouth of a user comprises capturing an image using a removable oral device inserted into the mouth of the user. For example, the palatal element comprises a camera within the body of the palatal element, and the camera is configured to capture the image of the material placed in the mouth of the user.

In another aspect, a method of monitoring athletic performance comprises measuring at least one athletic performance parameter of a user using a removable oral device inserted into a mouth of the user. For example, the palatal element comprises at least one electrode positioned within the body of the palatal element and exposed to fluid entering and exiting the mouth of the user. The at least one electrode can be configured to measure athletic performance from fluid entering and exiting the mouth of the user.

In an additional aspect, a method of monitoring intake of material into a mouth of a user comprises reading a bar code on material entering the mouth of the user using a removable oral device inserted into the mouth of the user. For example, the palatal element comprises a bar code reader within the body of the palatal element. The bar code reader can be configured to read a bar code on the material entering the mouth of the user.

In another aspect, a method comprises updating an electronic medical record of a user by providing the electronic medical record of the user from a memory unit within a removable oral device, and providing new medical information to the electronic medical record in the memory unit to update the electronic medical record.

In an additional aspect, a method of monitoring human health comprises measuring at least one physiological parameter of a user using a removable oral device inserted into a mouth of the user. For example, the palatal element comprises at least one electrode positioned within the body of the palatal element and exposed to fluid entering and exiting the mouth of the user. The at least one electrode can be configured to measure the physiological parameter from fluid entering and exiting the mouth of the user.

In another aspect, a method comprises monitoring head tremor comprising monitoring movement of the head using a removable oral device inserted into the mouth of the user. For example, the palatal element comprises an accelerometer within the body of the palatal element. The accelerometer can be configured to detect head movement to monitor the head tremor.

In an additional aspect, a method comprises monitoring head impact comprising monitoring movement of the head using a removable oral device inserted into the mouth of the user. For example, the palatal element comprises an accelerometer within the body of the palatal element. The accelerometer configured to detect head movement to monitor the head impact.

In another aspect, a method comprises monitoring chewing using a removable oral device inserted into the mouth of the user. For example, the palatal element comprises a piezoelectric sensor within the body of the palatal element. The piezoelectric sensor can be configured to measure pressure changes to monitor chewing.

In an additional aspect, a method comprising monitoring chewing using a removable oral device inserted into the mouth of the user. For example, the palatal element comprises a microphone configured to monitor chewing by measuring a change in decibel levels within the mouth or may comprise a pressure transducer positioned under a membrane or flexible portion of the palatal element.

In another aspect, a method comprises monitoring chewing using a removable oral device inserted into the mouth of the user. For example, the palatal element comprises a proximity sensor configured to monitor chewing by measuring a distance change between the proximity sensor and a sensor coupled to a lower tooth of the user.

In an additional aspect, a method comprises monitoring chewing of material entering a user's mouth using a removable oral device inserted into the mouth of the user. For example, the palatal element comprises a vibratory device configured to vibrate after the user has chewed a selected number of times to provide feedback to the user to swallow the chewed material.

In another aspect, a method comprises monitoring food intake volume entering a user's mouth using a removable oral device inserted into the mouth of the user. For example, the palatal element is configured to sense presence of the food in the user's mouth and absence of the food in the user's mouth as an indicator the food has been swallowed. The palatal element can be configured to provide feedback to the user once a selected number of swallows has been reached.

In an additional aspect, a method comprises monitoring material entering into a user's mouth comprising measuring nutrient species in the material using a removable oral device. For example, the palatal element comprises a processor and a memory unit electrically coupled to the processor. The palatal element may also comprise a sensor electrically coupled to the processor.

In another aspect, a method comprises monitoring material entering into a user's mouth comprising measuring nutrient species in the material using a removable oral device. For example, the palatal element comprises a processor and a memory unit electrically coupled to the processor. The palatal element may also comprise a multi-plexing sensor electrically coupled to the processor and configured to simultaneously measure caloric content of all the material entering into the user's mouth.

In an additional aspect, a method comprises providing real time measurements of material intake into a user's mouth using a removable oral device inserted into the mouth of the user. For example, the palatal element comprises a processor, a memory unit electrically coupled to the processor and a transmitter/receiver electrically coupled to the processor. The processor can be configured to transmit the recorded material intake to a site outside of the mouth of the user.

In another aspect, a method comprises monitoring blood pressure in a human comprising measuring blood pressure from a roof of the mouth of the human using a removable oral device. For examples, the palatal element comprises a processor, a memory unit electrically coupled to the processor and a pressure sensor electrically coupled to the processor.

In an additional aspect, a method comprises transferring information present on a removable oral device, the method comprising placing a removable oral device in contact with a case. For example, the palatal element comprises a processor, a memory unit electrically coupled to the processor and a transmitter/receiver electrically coupled to the processor. The case comprises a transmitter/receiver to receive the information from the transmitter/receiver of the oral device to transfer the information present on the removable oral device.

In another aspect, a method comprises determining a body fat percentage of a human comprising measuring bioelectrical impedance using a removable oral device. For example, the palatal element comprises an electrode. The body fat percentage can be determined using bioelectrical impedance and the electrode.

In an additional aspect, a method comprises using a removable oral device to transfer information from the removable oral device to a mobile device.

In another aspect, a method comprises transferring information from the removable oral device to a wearable device.

In an additional aspect, a method comprises providing a coaching platform configured for use with a removable oral device to assist in weight management of the user.

In another aspect, a method comprises providing a weight management platform configured for use with a removable oral device to assist in weight management of the user.

In an additional aspect, a method comprises providing a nutrition plan configured for use with a removable oral device to assist in weight management of the user.

In another aspect, a method comprises providing an exercise plan configured for use with a removable oral device to assist in weight management of the user.

Additional aspects, examples, embodiments and configurations are described further below.

### BRIEF DESCRIPTION OF THE SEVERAL VIEWS OF THE DRAWINGS

Certain configurations, aspects, embodiments and features are described with reference to the accompanying figures in which:

FIG. 1 is an illustration showing a generalized use of the removable oral device in weight loss/management applications in combination with a weight management application and/or coaching platform, in accordance with certain examples;

FIG. 2 is an illustration of a palatal element of a removable oral device, in accordance with certain examples;

FIG. 3 is another illustration of a palatal element of a removable oral device, in accordance with certain examples;

FIG. 4 is another illustration of a palatal element of a removable oral device, in accordance with certain examples;

FIG. 5 is an illustration showing a removable oral device comprising a palatal element and a clasping element installed in the mouth of a user, in accordance with certain configurations;

FIG. 6 is a perspective view of a removable oral device comprising a palatal element and a clasping element, in accordance with certain configurations;

FIG. 7 is a diagram showing use of the removable oral device in a weight management application, in accordance with certain examples;

FIG. 8 is a diagram showing usage monitoring of a removable oral device, in accordance with certain embodiments;

FIGS. 9A and 9B are diagrams showing transfer of usage values from a removable oral device to a mobile device, in accordance with certain examples; and

FIG. 10 is a diagram showing use of a removable oral device with a storage case and mobile device, in accordance with certain embodiments.

The particular configurations of the removable oral devices shown in the figures are not intended to be limiting. These configurations are shown and described merely to illustrate some of the various methods which can be implemented using one or more of removable oral devices.

### DETAILED DESCRIPTION

Various components are described below in connection with methods of using a removable oral device in weight management, athletic performance or other applications. The exact configuration of the removable oral device can vary depending on the intended use of the removable oral device. In certain examples, the removable oral device is not designed to alter a position of the user's teeth, e.g., is typically not configured and/or is not worn enough to alter the teeth position in a manner similar to braces or an orthodontic appliance. In certain embodiments, the removable oral device is not designed to retain a position of the user's teeth, e.g., does not function as an orthodontic retainer.

In certain embodiments, the removable oral devices described herein can be used in many different applications. For example, the removable oral devices can be used in weight management, e.g., weight control and/or weight loss applications, during exercise to monitor athletic performance, to monitor material intake, to measure physiological parameters, to monitor health, to track user health or activity, or for other uses. The removable oral device can be used in these and other applications with an on-board sensor or device or with an external sensor or device. Various systems where a removable oral device is used with an external sensor or device are described in more detail in the commonly assigned patent application bearing Ser. No. 62/477,766 and entitled “SYSTEMS INCLUDING REMOVABLE ORAL DEVICES.” Certain illustrative uses of the removable oral devices are described in more detail below. As noted below, the clasping element is optional and may or may not be present. In some configurations, a clasping element is present to facilitate removal of the removable oral device from the mouth.

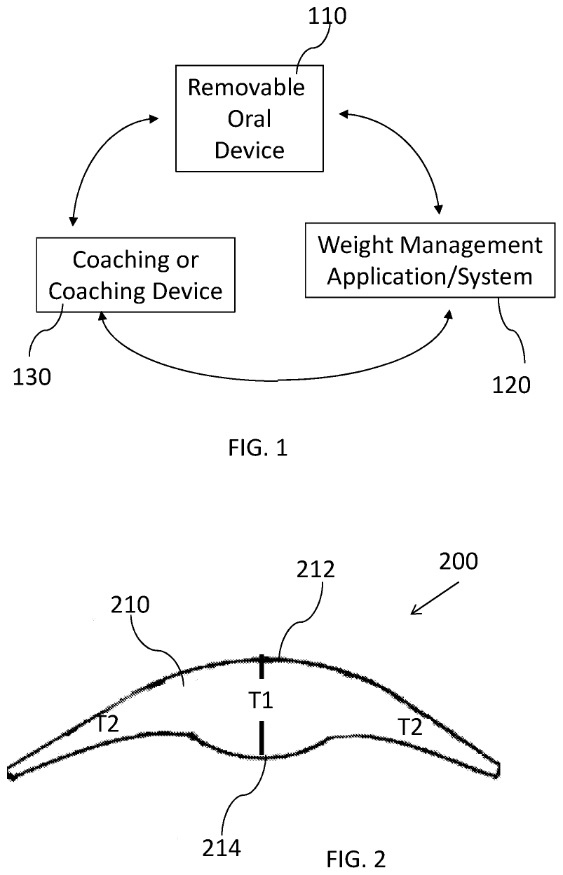

In certain examples, the removable oral device can be used in combination with a weight management application, e.g., a software application present on a mobile device, wearable device or other electronic device, and optionally in combination with one or more coaching platforms. A generalized illustration is shown in FIG. 1. A removable oral device 110 can be worn by a user during consumption of food. Information about usage of the removal oral device 110 during food consumption can be transferred to a weight management application/system 120 to permit the user to track their weight goals, food intake, device usage, etc. In some examples, a coaching platform 130, e.g., a live coach, in-person coach, pre-recorded coaching videos, audio, text messages or digital streams or the like, can be used in combination with the removable oral device 110 to enhance weight loss/management even further.

The exact removable oral device used in the methods described herein can vary. Numerous different types of removable oral devices are described in detail in commonly assigned patent application bearing Ser. No. 62/477,752 and entitled “REMOVABLE ORAL DEVICES.” One illustration of a removable oral device is shown in FIG. 2. The removable oral device comprises a palatal element 200 optionally coupled to one or more clasping elements (not shown). The palatal element generally comprises a suitable shape and materials to place an upper surface of the palatal element in contact with a roof of a user's mouth. The clasping element, when present, can be configured to assist in removal of the removable oral device from the user's mouth. In other configurations, the clasping element, when present, can be configured to engage some surfaces of the user's upper teeth, e.g., inner surface and/or outer surfaces, to assist in retention of palatal element in place when the removable oral device is inserted into the user's mouth. The palatal element 200 comprises a body 210 comprising a palatal surface 212 and a tongue surface 214. As noted in more detail in the application bearing serial number U.S. 62/477,764 and referenced above, the palatal element 200 can be produced using molding, printing or other suitable techniques. Notwithstanding that many different methods and devices can be used to produce the palatal element 200, the palatal element 200 is generally designed so the palatal surface 212 conforms or contours to the roof of a user's mouth. A tongue surface 214 generally mirrors or replicates the roof or palate shape of the user. The palatal element 200 has a thickness T1 (from the highest point or apex of the palatal surface 212 to the lowest point of the tongue surface 214) at a medial portion of the palatal element 200. The thickness T2 at an edge of the palatal element 200 is generally less than the thickness T1. For example, the portions of the palatal element 200 which sit against or are adjacent to the upper teeth are typically 2×, 3×, 4× or 5× less thick than the thickness T1 to provide a more comfortable fit in a user's mouth. As noted in more detail herein, the thickness T1 may be adjusted or adjustable as desired. Without wishing to be bound by any particular theory or configuration, the thickness T1 can be selected to decrease the overall oral volume of the mouth available for chewing and/or to better position one or more sensors (not shown) of the palatal element 200 in an appropriate position. The amount by which the oral volume is reduced is controlled generally by the thickness T1 and may vary from user to user or depending on the particular disorder to be treated, the particular condition to be monitored and other criteria. While the exact reduction in volume can vary, in some examples, the thickness T1 is selected such that the oral volume is reduced by 5% to about 50% (as compared to an original oral volume where no removable oral device is present), more particularly reduced by about 15% to about 35% or about 25% to about 35%.

In certain embodiments, the removable oral device comprising the palatal element 200 can be used to reduce the overall volume of the mouth to slow food intake. For example, the decrease in overall volume provided when the palatal element 200 is inserted into a user's mouth permits smaller bites of food and/or lower overall food volume per bite to be introduced into the mouth. This result can increase the overall time it takes to ingest a particular volume of food, which can promote increased satiety and an overall reduction in food intake volume, e.g., fewer overall calories are consumed when the removable oral device is present compared to the removable oral device not being inserted into a user's mouth.

In some instances, the removable oral device comprising the palatal element 200, e.g., one without a clasping element or one with a clasping element, can be used in weight management. For example, a user can insert the removable oral device prior to eating, e.g., once per day, twice per day, three times per day, once per week, five days per week, twice per week, at each meal, at each meal and at each snack or every time a user ingests food to assist in weight management. As noted herein, when the removable oral device is in place, the overall level of calories ingested during a particular eating session can be reduced, which can result in weight loss and/or weight management. In certain configurations, the removable oral device can be used in combination with a coaching platform or coaching based devices or media to provide feedback and/or monitoring of the user's use of the removable oral device and/or to assist in weight management. Such coaching platforms may take the form of in-person sessions, external sessions over a remote connection or automated sessions, text messages, videos, etc. retrieved by the user through one or more software applications on a mobile device, wearable device or other electronic device.

In some instances, the removable oral device comprising a palatal element can be used until a user's body fat percentage or body mass index (BMI) reaches a desired level. For example, methods of using the removable oral device may comprise identifying a human subject with a body mass index between 25 to 35 or between 27 to 35 or between 25 to 32. If desired, the removable oral device could also be used with people whose body mass index exceeds 30 or people whose body mass index is under 25. In some examples, the removable oral device can be used with human males comprising a body fat percentage between 22-29% or exceeding 26% or with human females comprising a body fat percentage of 31-39% or exceeding 31%. In some examples, the removable oral device use frequency can be reduced once the user's BMI or body fat percentage drops below a selected level, e.g., below a BMI of 25 or below a body fat percentage of 22% or 25% for human males and 31% for human females. For example, weight maintenance can be attained by using the removable oral device once per week or 2-3× per week rather than using the removable oral device daily to assist in weight loss.

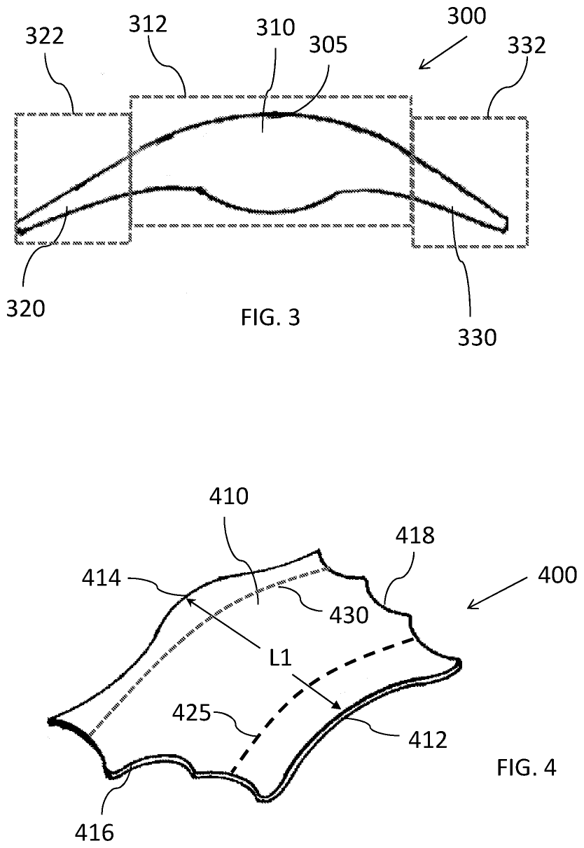

In certain examples and referring to FIG. 3, a palatal element 300 is shown that comprises areas 310, 320 and 330. Area 310 is designed to provide a desired thickness to reduce the overall oral volume and comprises an apex 305, which is typically the highest point of the palatal element 300. Areas 320 and 330 are designed to be positioned adjacent to certain teeth and assist in retention of the removable oral device in place. In certain configurations, the material present in area 310 (as shown by box 312) may be harder, e.g., may have a higher Vickers hardness value (HV), than the materials present in areas 320, 330 (as shown by boxes 322 and 332). While the exact methodology used to determine a HV can vary, suitable methods typically involve indenting the test material with a diamond indenter under a selected load, e.g., 30 kg of force (kgf), and measuring the depth of the indentation. The indentation depth can be correlated to materials hardness using suitable lookup tables or calibration curves. Commercial instruments to determine Vickers hardness values are available from Shimadzu (Japan) and LECO (Japan). In some instances, the protocols described in ASTM E384-16 entitled “Standard Test Method for Microindentation Hardness of Materials” can be followed to determine Vickers hardness values. The hardness/softness of the materials at areas 320, 330 can be the same or can be different. In some examples, areas 320, 330 comprise softer materials (compared to the hardness of the material at area 310) to permit these areas to function, at least to some degree, as a seal or gasket that prevent foods or other materials from entering into any space between a palatal surface of the palatal element and the roof of the user's mouth. Depending on the particular materials used, the hardness at the areas 320, 330 can be at least 2×, 3×, 4× or 5× less than the hardness at area 310. For example, the Vickers hardness value (HV) at areas 320, 330 can be at least 5% less, at least 10% less, at least 20% less, at least 30% less, at least 40% less or at least 50% less than the HV at the area 310. The hardness at area 310 is generally less than that of glass or hard plastics such that the area 310 can flex to some degree during chewing of food. For example, in some instances, the HV value at the apex 305 may be 20 HV or more, whereas the HV value at the edges 320, 330 can independently be less than 20 HV. In other examples, the HV value at the apex 305 can be 10 HV or more and the value at the edges 320, 330 can be less than 10 HV. In other configurations, the HV value at the apex 305 can be 10 HV or more and the value at the edges 320, 330 can be less than 5 HV. The exact level of hardness used may vary from subject to subject. For example, certain individuals may find hard apex areas to be uncomfortable, and the material hardness at the apex can be lowered for those subjects by, for example, reducing the level of cross-linking in the polymeric materials used to produce the palatal element 300.

In some embodiments, the material used at the edges 320, 330 may be the same or may be different than material present at the apex 305 of the palatal element. For example, the material at the edges can be the same material but it may be cross-linked to a lesser degree to be softer than the material at the apex section of the palatal element. In other examples, a different material is used for the edges and is coupled to other material of the palatal element through one or more cross-linkers. Where two different materials are used, there is generally no discernible interface between the materials which might be obtrusive or uncomfortable.

In some embodiments, the material present in the palatal element may be an acrylic, a polycarbonate, a polyolefin, a thermoplastic polymer, a thermoset polymer or combinations thereof. If desired, the material may comprise elastomers, elastomeric fibers or other materials to alter the overall hardness of one or more areas of the palatal element. The materials can be cross-linked or cured by sprinkling or mixing a cross-linker with the material either pre-use or post-use. For example, a mold of the user's mouth can be used with the material to provide a palatal element. The material can be added to the mold and then cross-linked by sprinkling a cross-linker onto the added material in the mold. Other materials, methods and processes for producing the palatal element are described in more detail in the commonly assigned application bearing Ser. No. 62/477,764, which is incorporated herein by reference. In some examples, the palatal surface and/or tongue surface can be smooth to prevent food from sticking to the removable oral device. If desired, however, the surface may be bumpy or comprise ridges or other features to mimic the tongue feel when the tongue is placed against the roof of the user's mouth. In other instances, the palatal element may comprise one or more coatings including, but not limited to, anti-bacterial coatings, non-stick coatings or coatings which may impart color or ornamental designs to the palatal element. In some examples, the material used to produce the palatal element can be a combination of two or more acrylics. For example, polymethacrylate can be used at or near the apex 305 and Silident™ acrylic polymers can be used at the edges 320, 330 of the palatal element 300. If desired, there may be a transition zone between the harder and softer areas which comprises an intermediate hardness from the presence of both materials, e.g., is less hard than the apex surface but is harder than the edges.

In certain examples, a perspective view of a palatal element is shown in FIG. 4. The palatal element 400 comprises a body 410 with sides 412, 414, 416 and 418. Side 412, e.g., an anterior side, generally is adjacent and/or contact inner surfaces of the anterior teeth. Side 414, e.g., a posterior side, is positioned in the back of the mouth when the removable oral device is inserted. Side 416 is positioned adjacent to the inner surfaces of tooth numbers 1-4 (or 2-4 when the wisdom teeth have been removed) when the removable oral device is inserted. Side 418 is positioned adjacent to the inner surfaces of tooth numbers 13-16 (or 13-15 when the wisdom teeth have been removed) when the removable oral device is inserted. The length L1 of the body 410 may vary and is generally designed to be large enough so the body 410 provides a desired oral volume reduction but is not so large that a user may gag or have difficulty breathing when the removable oral device is inserted.

In some examples, some portion or the anterior side and/or posterior side can be removed to facilitate a better user experience with the removable oral device. For example, a volume of the palatal element toward the anterior side 412, e.g., the volume from the line 425 forward toward the anterior side 412 can be removed or reduced to reduce lisping. In certain embodiments, about 1-10% of the volume from the anterior side 412 can be removed to assist in reduction of lisping when the removable oral device is in place. If desired, a crescent shape (or other shape) may be provided at the anterior side 412 to reduce lisping. In some examples, trimming the posterior side 418 of the device in a crescent shape can be performed to eliminate or reduce contact with the soft palate to address individuals with heightened gag reflex. For example, a posterior volume from line 430 toward the posterior side 414 can be removed to reduce the likelihood of gagging when the removable oral device is present in the mouth. In some examples, a crescent shape or other non-linear shape may be provided at the posterior side to reduce gagging. In certain embodiments, about 1-15% of the volume from the posterior side 414 can be removed to assist in reducing the likelihood of gagging. As noted in more detail in U.S. 62/477,764, the palatal element can be produced using printing, molding, etc. from a digital scan or from an impression mold of the user's mouth. A palatal element can then be trimmed or shaped as desired to provide a desired overall volume reduction while at the same time minimizing or reducing the likelihood of lisping and/or gagging.

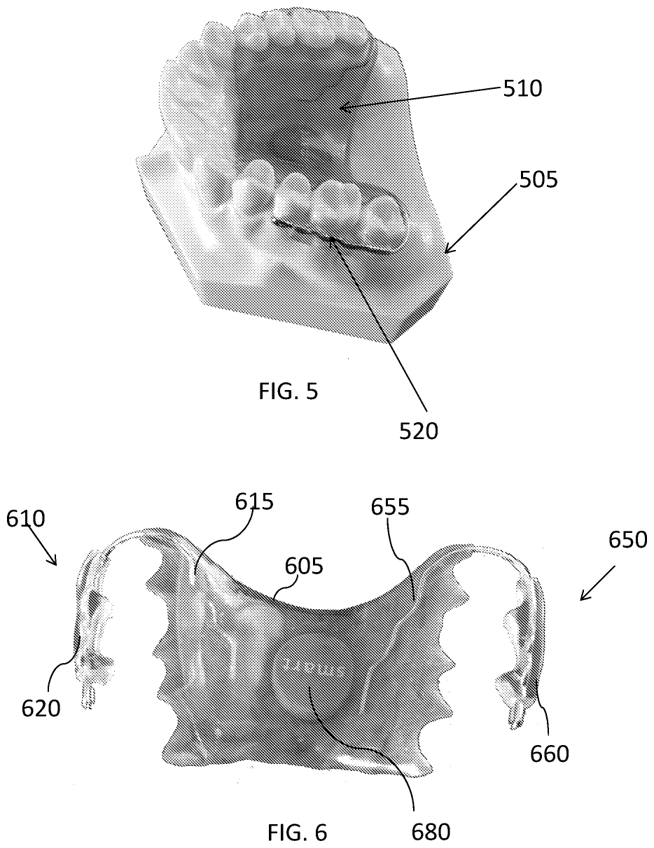

In certain configurations, the removable oral devices described herein also comprise a clasping element. As noted herein, the clasping element is optional but may be present to assist removal of the palatal element from the user's mouth. Referring to FIG. 5, a perspective view of a removable oral device 500 comprising a palatal element 510 and a clasping element 520 is shown positioned around a tooth mold 505. The clasping element 520 is generally configured with a support element or wire that is embedded within material such that the wire itself does not directly contact the outer surfaces of the teeth. While the clasping element may be configured as a wire or wires in some instances, in other cases the clasping element may comprise a plastic or flexible moldable material etc., or other non-metal based materials. In some examples, the clasping element may comprise chromium-nickel alloys such as, for example, Elgiloy™ materials which are cobalt-chromium-nickel alloys. In other examples, the clasping element may comprise titanium, titanium alloys, nickel titanium materials such as Nitinol, etc. In some embodiments, the material of the clasping element is generally inert so that it does not tarnish, rust, corrode or otherwise degrade during use of the removable oral device. In certain configurations, some portion of the wire may directly contact the rear surface of the back teeth to assist in retention of the palatal element 520 against the roof of a user's mouth. The wire may be a continuous wire which runs from one side of the palatal element 510 to the other or two or more separate wires can be present with one wire being present in a respective clasping element. For example and referring to FIG. 6, a clasping element 610 comprises a wire 615 and associated material 620 on some portion of the wire. Another clasping element 650 comprises a wire 655 and associated material 660 on some portion of the wire 655. Some portion of the wires 615, 655 is embedded within a body of a palatal element 605. The material 620, 660 generally rests against outer surfaces of the teeth when the palatal element 605 is engaged to the roof of the mouth. The material 620, 660 is generally a softer material, e.g., has a lower HV, than the material present in the apex of the palatal element 605. The wires 615, 655 can assist in removal of the removable oral device from the mouth and/or assist in retaining the removable oral device in place. For example, surface tension between a palatal surface of the palatal element 605 and the roof of a user's mouth can “lock” the palatal element to the roof of the mouth. The wires 615, 655 can provide leverage to assist in breaking of the surface tension and removal of the device from the mouth. An optional usage sensor 680 (discussed in more detail below) is also shown as being embedded in the palatal element 605. While two clasping elements 610, 650 are shown in FIG. 6, only a single clasping element may be present if desired or, no clasping element may be present at all.

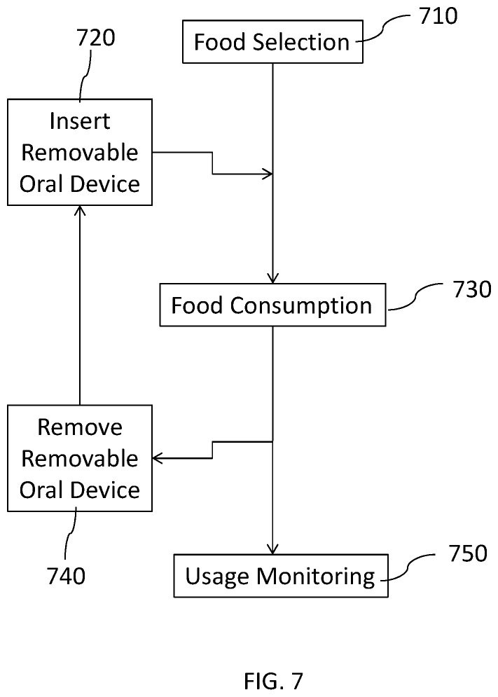

In certain instances, the removable oral device can be used in a method of promoting weight loss and/or weight management. The methods of using the removable oral device are typically non-surgical methods, which provide desirable attributes over conventional surgical techniques commonly used to promote weight loss. An overview of this usage is shown in FIG. 7. A user selects food (and/or liquids) to eat during a step 710. Prior to consumption of the food (and/or liquids), the user inserts a removable oral device into their mouth at step 720. The user then consumes the food (and/or liquids) with the removable oral device inserted at step 730. As noted below, a usage sensor can be present in or on the removable oral device to sense when the device has been inserted into the user's mouth. The presence of the removable oral device in the oral cavity during meal consumption slows food intake and can reduce the overall caloric intake during the meal. Once the food has been eaten, or once the user decides they are full, the removable oral device is removed at a step 740 and usage monitoring of the removable oral device 750 can be performed on a selected basis, as discussed in more detail below. Depending on the user's weight goals and other factors, the same removable oral device can be inserted, for example, daily during every eating period, every other meal or one meal per day. If desired, different removable oral devices can be used at different meals. For example, it may be desirable to reduce caloric intake to a greater degree later in the day to place the user's metabolism in a “fasting” state overnight. A removable oral device that reduces the oral volume to a much greater degree can be used later in the day in such scenarios.

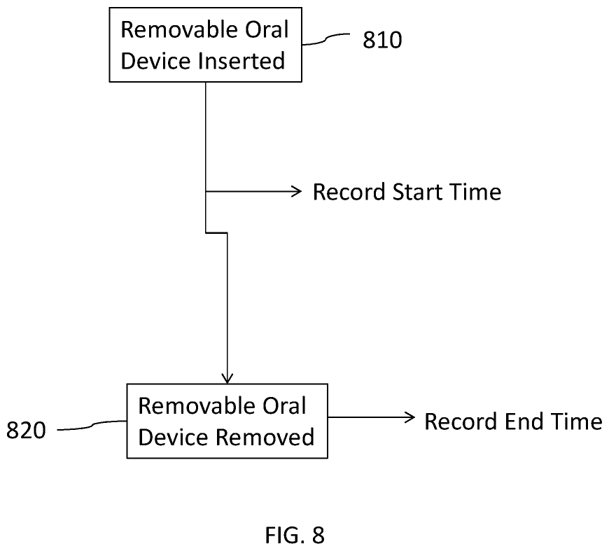

In certain embodiments, the removable oral device may comprise a usage sensor which can sense when the removable oral device has been inserted into the user's mouth. The usage sensor can be used to monitor use frequency of the removable oral device. For example, a usage sensor can be used to log the duration which the removable oral device is present in a user's mouth or can be used to simply log the device has been inserted into the oral cavity without monitoring the exact duration of usage. Referring to FIG. 8, a flow chart shows that after a removable oral device is inserted at step 810, an event or log entry can be recorded as a measure of the start time of usage of the removable oral device. Recording of a start time may be performed by itself as a measure that the removable oral device is present. If desired, however, the duration of usage of the removable oral device at each meal can be recorded by recording an even of log entry when the removable oral device is removed at a step 820. The difference in start and end time is used to determine the number of minutes the removable oral device was present during a meal, and by extension, how long it took the user to consume the food during that meal. This information can be transferred to the application software/system as noted in more detail below. The usage sensor may comprise a timing circuit, a timer or other suitable devices such as, for example, a processor, to monitor the duration the removable oral device is present in the oral cavity. If desired, the removable oral device may also comprise a clock to permit correlation of usage of the removable oral device with the time of day. Additional suitable electrical components may also be present on or in the removable oral device as noted in more detail in commonly assigned patent application bearing Ser. No. 62/477,766.

In certain embodiments, the exact nature of the usage sensor can vary depending on the desired configuration of the removable oral device. For example, the usage sensor may be a camera which captures images of food entering the mouth. The usage sensor may comprise an electrode which can sense the presence of nutrients in the food, e.g., carbohydrates, proteins, lipids, etc. or can sense a change in conductance due to the presence of solid food in the oral cavity. The usage sensor may comprise a temperature sensor, thermocouple or similar device to sense a temperature change after the removable oral device is inserted into the oral cavity. In other instances, the usage sensor may comprise a bar code reader which can be used to read one or more bar codes indicative of the meal time, e.g., breakfast, lunch, dinner, etc. or to read bar codes printed on food or added to food or present on packaging associated with the food. In other examples, the usage sensor may comprise an accelerometer which can be used to monitor usage based on movement of the mouth up and down during chewing. In further instances, the usage sensor may comprise a piezoelectric sensor or pressure sensor which can sense changes in pressure applied to the removable oral device by food being present in the mouth during chewing. In further examples, the usage sensor may comprise a microphone which can detect chewing sounds as a measure of usage of the removable oral device. In other configurations, the removable oral device may comprise a proximity sensor which can communicate electrically with a crown, filling, mouth insert, etc. on a tooth (or present in another portion of the oral cavity) as a measure of the device being present in the mouth. In further examples, one or more of a vibratory device, an optical sensor, a processor, a memory unit, a transmitter, a receiver, a transmitter/receiver, an antenna or other electrical components may also be present in or as part of the usage sensor.

In certain embodiments, usage monitoring of the removable oral device may be measured using hardware and/or software applications. In one example, the usage monitoring may be recorded using a processor such as those described below. In additional configurations, the usage monitoring can be implemented by transferring the recorded events on the removable oral device from the removable oral device to application software present on a mobile device, wearable device, laptop, computer or other electronic device. The electronic device is typically separate from the removable oral device, though if desired the electronic device could be integral to the removable oral device and another device could be used simply to display the information present on the removable oral device. For example, a mobile device may be held adjacent to the removable oral device to wirelessly receive information from the removable oral device and display it on the screen of the removable oral device.

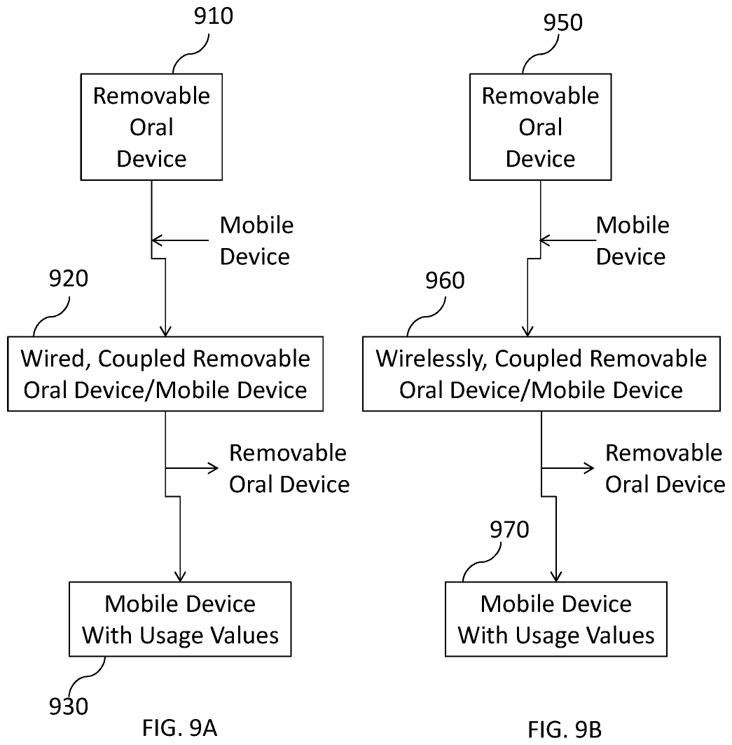

In certain examples and referring to FIG. 9A, a process is shown where usage values are transferred from the removable oral device to a mobile device in a wired manner. At step 910, the removable oral device is coupled to a mobile device in a wired manner, e.g., using a USB cable, lightning cable or other hardwired cables/interfaces. Usage values are then transferred from the removable oral device to the hardwired, coupled mobile device at a step 920. The removable oral device is then decoupled from the mobile device to provide a mobile device with transferred usage values at a step 930. In some examples, it may be desirable to wirelessly transfer the usage values from the removable oral device to the mobile device. Referring to FIG. 9B, at a step 950 a removable oral device is wirelessly coupled to a mobile device, e.g., using an optical device such as an infrared transmitter, a Bluetooth device on the removable oral device and the mobile device, using a cellular chip on the removable oral device or using near field communication devices. Usage values are then transferred wirelessly from the removable oral device to the hardwired, coupled mobile device at a step 960. The removable oral device is then wirelessly decoupled from the mobile device to provide a mobile device with transferred usage values at a step 970. While not shown, a dongle, interface or other device can be coupled to the mobile device in a hardwired or wireless manner and used as a transfer interface to transfer usage values from the removable oral device to a mobile device. Further, one or more integral components of the mobile device, e.g., the camera, could be used to read optical signals from the removable oral device and convert those into usage values. The mobile device, removable oral device, dongle or interface may implement suitable encryption/decryption methods to prevent the usage values of a particular human subject from being viewed by an unauthorized individual. For example, usage values transmitted from the removable oral device may be encrypted prior to transmission to a mobile device, and the mobile device (once authenticated or including a suitable decryption key), can decrypt the values for further use.

In some examples, the removable oral device can be used in combination with a storage case to transfer usage values from the removable oral device to a mobile device in a wired or wireless manner. For example, the storage case can interface with the removable oral device in a wired or wireless manner. The coupled oral device/storage case can then be used to transfer usage values from the coupled removable oral device/storage case to a mobile device. While not wishing to be bound by any particular configuration, to minimize the number of electrical components present in or on the removable oral device and to reduce overall power consumption by the removable oral device, it may be desirable to include certain components in the storage case rather than on or in the removable oral device itself. While the removable oral device typically comprises a power source such as a battery, the power source may not be rechargeable or may provide only a limited amount of power between charges. By moving some of the electrical components to the storage case, the overall power consumption by the removable oral device can be reduced. In some instances, the removable oral device may comprise a usage sensor, a power source and a transmitter which can provide usage values to the storage case, e.g., in a wired manner or using wireless devices such as an infrared light, a Bluetooth device, a radio frequency transmitter or a cellular chip. The storage case is typically rechargeable by way of a wired or wireless charging method and may include a replaceable power source such as a battery, fuel cell or other power sources.

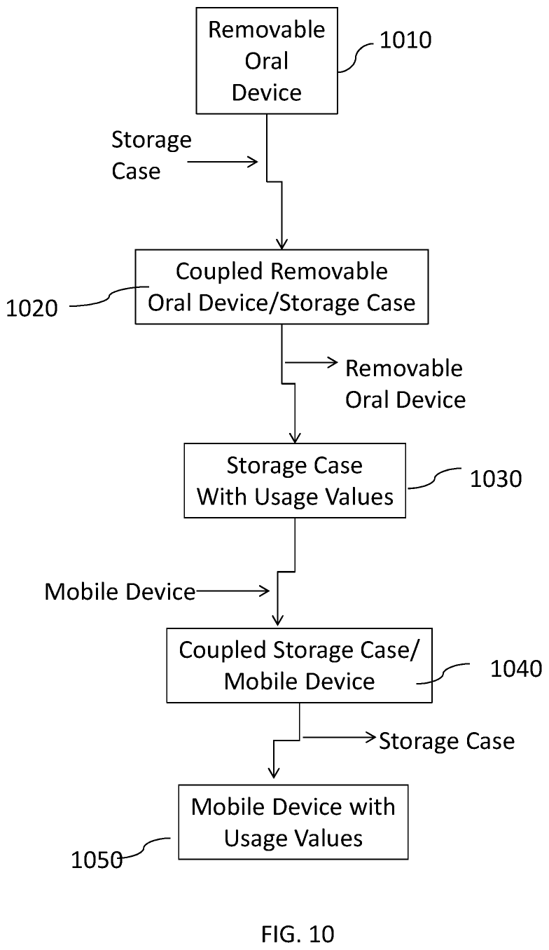

In certain examples and referring to FIG. 10, in use of the removable oral device in combination with a storage case, the removable oral device is placed into the storage case at a step 1010 to provide a coupler removable oral device/storage case at a step 1020. The storage case may comprise a processor, transmitter/receiver or other components to receive usage values from the removable oral device. The removable oral device and storage case can be paired such that only usage values from a single paired removable oral device are read by the storage case. Values from an unpaired removable oral device are not read or are otherwise discarded. Once the usage values are transferred to the storage case, then the removable oral device can be removed to provide a storage case with the usage values at a step 1030. If desired, however, the removable oral device may remain in the case for storage. For example, it is not necessary to remove the removable oral device from the storage case prior to transfer of the usage values from the storage case to a mobile device. The storage case can then be coupled to a mobile device at a step 1040. The usage values can be transferred from the storage case to the mobile device, and the storage case and mobile device can be decoupled at a step 1050 to provide a mobile device with the usage values. While not shown, the storage case typically comprises a processor, one or more memory units and a Bluetooth receiver/transmitter to provide usage values from the case to a mobile device, wearable device or other electronic device.

In certain configurations, the mobile device which receives the usage values from the removable oral device or storage case may be remote from the removable oral device or storage case. For example, the removable oral device or storage case or both may comprise a cellular chip which can transmit the usage values over a cellular network to a remotely located computer system. This transmission can occur in place of or in addition to local transmission to a mobile device. For example, it may be desirable to simultaneously transmit the data to a local mobile device and to a remote system to permit a remote coach to access the usage values. Where usage values are transmitted, they can be transmitted and used by application software to permit monitoring/tracking of a user's weight, diet, weight goals, dietary goals and the like. The application software can be configured to display weight trends, caloric intake trends (by hour or by day or by month, etc.) or other information desirable for maintaining weight.

In one configuration of application software suitable for use with the removable oral device, the application software may display the number of meals eaten, the number of meals eaten with the removable oral device inserted, user information (height, current weight, target weight, etc.), weight trends (in tabular or graphical formats), caloric intake, exercise schedules, and other items. The application software can be used to provide for small or mini-behavioral modifications to enhance or promote weight management. As noted in more detail below, the application software can be used in combination with a coach or a coaching platform to further enhance or promote weight management.

In certain instances, a user's goals and current weight management plans can be aided by way of a coach, e.g., a human person who reviews information on the application software and provides feedback to the removable oral device user, or an automated coaching platform, e.g., which can use smart algorithms and select automated audio, video or text messages to provide to the removable oral device user to assist in their weight management. The coaching/coaching platform can recommend dietary changes, provide exercise schedules, nutrition information and/or otherwise provide minor and gradual behavioral modification to promote weight loss and/or weight management. The coaching/coaching platform can be implemented in phases including an interventional phase, e.g., to promote weight loss, and a maintenance phase, e.g., to maintain current weight. Removable oral device usage frequency can be altered by the coach or coaching platform depending on the particular phase and goals of the user. While not required, the interventional phase can last from 0 to 6 months with the maintenance phase lasting beyond the 6 month initial period. Where the removable oral device is used with severely obese individuals (or those with metabolic issues), the interventional phase can be implemented for a period longer than 6 months if desired.

In some examples, the coaching platform can be integrated with the application software such that audio, video or text messages are automatically provided to the user when they transfer the usage values to the mobile device, wearable device, etc. For example, based on the weight loss trends, food intake trends, etc., the application software can retrieve suitable messages and provide them through the application software to guide the user in their weight loss or weight management. The application software and/or messages may be designed such that the mobile device is “locked out” until the entire message is played, which increases the chance the user will hear and/or watch the retrieved messages.

While an automated coaching platform may be desirable in some instances, the exact same feedback can also be implemented by in-person sessions, phone sessions, video sessions, etc. using a live coach. The live coach can perform analyses of the usage values and guide the removable oral device user in connection with removable oral device usage, food intake, exercise schedules and the like. While not wishing to be bound by any particular theory, certain users may benefit more from live coaching rather than automated messages. In addition, live coaching can be sought out by those users who are serious about weight management, since live coaching feedback typically involves two way communication between the removable oral device user and the coach and permits the user to ask questions or otherwise seek specialized advice which may not be available through an automated system.

In certain embodiments, the removable oral devices described herein can be used in methods of monitoring intake of material into a mouth of a user. For example, the method may comprise capturing an image using a removable oral device inserted into the mouth of the user. As noted herein, the removable oral device comprises a palatal element coupled to a clasping element, wherein the palatal element is configured to contact a roof of a user's mouth at a palatal surface. The palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both if desired) comprises a camera within the body of the palatal element. The camera is configured to capture the image of the material placed in the mouth of the user. If desired, the palatal element may comprise a variable hardness, e.g., the edges of the palatal element can be softer than an apex surface of the palatal element. For examples, the method comprises configuring the removable oral device to comprise a variable hardness across the tongue surface of the body. In other embodiments, the method comprises configuring the removable oral device with a rechargeable power source electrically coupled to the camera, e.g., a battery, fuel cell, etc. In further examples, the method comprises transmitting the image captured by the camera. As noted herein, the image can be transmitted to a storage case or directly to a mobile device. In other embodiments, the method comprises transmitting the image wirelessly. In some instances, the method comprises automatically switching on the camera in response to a sensed temperature change. In other examples, the method comprises automatically switching the camera off in response to a second sensed temperature change. In some instances, the method comprises storing the captured image in a memory unit present in the removable oral device. In some examples, the method comprises capturing the image through an optical window present in the body. In further instances, the method comprises configuring the removable oral device to not alter a position of the user's teeth. In certain embodiments, the method comprises configuring the removable oral device to not retain a position of the user's teeth. In some examples, the removable oral device comprising the camera can be used with a processor, memory unit, etc. as noted in more detail below.

In one configuration, a method comprises monitoring chewing using a removable oral device inserted into the mouth of the user. The removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both) comprises a piezoelectric sensor within the body of the palatal element, wherein the piezoelectric sensor is configured to measure pressure changes to monitor chewing.

In certain embodiments, the method comprises configuring the piezoelectric sensor to automatically switch on when the palatal element reaches a selected temperature. In some examples, the method comprises configuring the palatal element with a device configured to provide the user sensor feedback after the piezoelectric sensor measures a selected number of chews. In certain examples, the method comprises configuring the sensory feedback as an audible alert or as a vibration. In some embodiments, the method comprises configuring the palatal element with a second piezoelectric sensor. In other embodiments, the method comprises configuring the removable oral device to not alter a position of the user's teeth. In some instances, the method comprises configuring the removable oral device to not retain a position of the user's teeth. If desired, a removable oral device with a piezoelectric sensor may comprise a variable hardness across a palatal surface of the device.

In some configurations, the method comprises monitoring chewing using a removable oral device inserted into the mouth of the user. The removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element comprises a microphone configured to monitor chewing by measuring a change in decibel levels within the mouth.

In certain examples, the method comprises configuring the microphone with a speaker to provide a sensory feedback after the change in decibel levels has been measured for a selected period. In other examples, the method comprises configuring the sensory feedback as an audible alert or as a vibration. In some embodiments, the method comprises configuring the palatal element with a second microphone. In certain examples, the method comprises configuring the palatal element with a recording medium to record audio. In some instances, the method comprises configuring the removable oral device to not alter a position of the user's teeth. In certain examples, the method comprises configuring the removable oral device to not retain a position of the user's teeth. If desired, a removable oral device comprising a microphone may comprise a variable hardness across a palatal surface of the device.

In another configuration, a method comprises monitoring chewing using a removable oral device inserted into the mouth of the user using a proximity sensor. The removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element comprises a proximity sensor configured to monitor chewing by measuring a distance change between the proximity sensor and a sensor coupled to a lower tooth (or some other anatomical feature in the mouth) of the user.

In certain examples, the method comprises configuring the proximity sensor to measure contact of the proximity sensor to the sensor coupled to the lower tooth of the user. In other examples, the method comprises configuring the proximity sensor to detect a radio wave emitted by the sensor coupled to the lower tooth of the user. In some examples, the method comprises configuring the proximity sensor to detect light emission emitted by the sensor coupled to the lower tooth of the user. In some embodiments, the method comprises configuring the proximity sensor to detect an ultrasound wave emitted by the sensor coupled to the lower tooth of the user. In other examples, the method comprises configuring the removable oral device to not alter a position of the user's teeth. In additional examples, the method comprises configuring the removable oral device to not retain a position of the user's teeth. If desired, a removable oral device with a proximity sensor may comprise a variable hardness across a palatal surface of the device.

In certain examples, a method monitoring chewing of material entering a user's mouth using a removable oral device inserted into the mouth of the user with a vibratory device. In some examples, the removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both) comprises a vibratory device configured to vibrate after the user has chewed a selected number of times to provide feedback to the user to swallow the chewed material.

In certain instances, the method comprises configuring the palatal element with a chewing sensor electrically coupled to the vibratory device. In some embodiments, the method comprises configuring the clasping element with a chewing sensor electrically coupled to the vibrator device. In other examples, the method comprises configuring the removable oral device to not alter a position of the user's teeth. In some instances, the method comprises configuring the removable oral device to not retain a position of the user's teeth. The chewing sensor, for example, may be designed to monitor movement of the mouth between positions, e.g., clenched teeth and unclenched teeth, as a measure of chewing. If desired, a removable oral device with a vibratory device may comprise a variable hardness across a palatal surface of the device.

In some examples, the coaching platform can be present as application software, e.g., present on a mobile device or a computer. For example and referring to FIG. 2, a removable oral device 210 can be used with a mobile device 220 comprising coaching application software. The mobile device may be, for example, a cellular phone, a laptop computer, a tablet, a wearable device such as a Fitbit® device, an Apple Iwatch device or the like. In some examples, the coaching application software comprises menus or prompts to permit a user to navigate through the software based on the user's use of the removable oral device. Based on a user's desired weight loss and/or weight management goals, different feedback may be provided by the coaching platform to assist the user in obtaining their weight management goals. If desired, the coaching platform may be used in combination with a nutrition plan, an exercise plan or other schedules or plans. In some instances, the removable oral device can be provided along with the coaching platform or may be provided separately from the coaching platform.

In some configurations, a method comprises providing a weight management platform configured for use with the removable oral device to assist in weight management of the user. The weight management platform may comprise an eating schedule, caloric intake schedule, food schedule, nutrition plan, exercise plan, etc. The weight management plan may also be configured to specify removable oral device usage schedules, e.g., once per day, once per week, once per day for 5 days per week, at every meal, etc. If desired, the weight management plan can be based on information transferred from the removable oral device. In some instances, the removable oral device can be provided along with the weight management plan or may be provided separately from the weight management plan.

In other configurations, a method comprises providing a nutrition plan configured for use with the removable oral device to assist in weight management of the user. The nutrition plan, for example, can be tailored or tuned based on information transferred from the removable oral device and/or the user's weight goals. The nutrition plan may comprise pre-packaged food such as that provided by Weight Watchers or Jenny Craig programs. The nutrition plan can be altered weekly, monthly or at other schedules based on the user's goals and/or information from the removable oral device. The nutrition plan can be used in combination with a coaching platform and/or other plans or platforms. In some examples, the removable oral device can be provided along with the nutrition plan or may be provided separately from the nutrition plan.

In another configuration, a method comprises providing an exercise plan configured for use with the removable oral device to assist in weight management of the user. The exercise plan, for example, can be tailored or tuned based on information transferred from the removable oral device and/or the user's weight goals. The exercise plan may comprise pre-selected exercise routines or programs. The exercise plan can be altered weekly, monthly or at other schedules based on the user's goals and/or information from the removable oral device. The exercise plan can be used in combination with a coaching platform and/or other plans or platforms. In some examples, the removable oral device can be provided along with the exercise plan or may be provided separately from the exercise plan.

In some instances, the removable oral devices described herein can be used in applications other than weight management. Where non-weight management applications are performed using the removable oral device, the palatal element may have a certain thickness to accommodate the electrical device or component but may not generally reduce the overall oral volume to a substantial degree. For example, where the removable oral device is used in non-weight management applications, the overall oral volume can be reduced by 20% or less or by about 15% to about 1%. In some instances, the thickness of the palatal element is sufficient to encompass or embed the electrical component within the palatal element such that the electrical component is not exposed to liquids or solids entering into the mouth.

In other configurations, a method of monitoring athletic performance comprises measuring at least one athletic performance parameter of a user using a removable oral device inserted into a mouth of the user. As noted herein, the removable oral device comprises a palatal element coupled to a clasping element, wherein the palatal element is configured to contact a roof of a user's mouth at a palatal surface. The palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or the clasping element or both) comprises at least one electrode positioned within the body of the palatal element and exposed to fluid entering and exiting the mouth of the user, the at least one electrode configured to measure athletic performance from fluid entering and exiting the mouth of the user.

In some examples, the method comprises configuring the body with a memory unit configured to store the measured athletic performance. In other examples, the method comprises configuring the electrode to measure one or more of oxygen, carbon dioxide and water as the measure of athletic performance. In further examples, the method comprises configuring the removable oral device with a rechargeable power source electrically coupled to the at least one electrode, e.g., a battery or fuel cell. In some embodiments, the method comprises transmitting the measured athletic performance wirelessly to an external device. In other examples, the method comprises automatically switching on the electrode in response to a sensed temperature change. In certain examples, the method comprises automatically switching the electrode off in response to a second sensed temperature change. In other examples, the method comprises configuring the removable oral device to not alter a position of the user's teeth. In certain embodiments, the method comprises configuring the removable oral device to not retain a position of the user's teeth. If desired, a removable oral device used in monitoring athletic performance may comprise a variable hardness across a palatal surface of the device.

In another configuration, a method of monitoring intake of material into a mouth of a user comprises reading a bar code on material entering the mouth of the user using a removable oral device inserted into the mouth of the user. For example, the removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both) comprises a bar code reader within the body of the palatal element. The bar code reader can be configured, for example, to read a bar code on the material entering the mouth of the user.

In certain examples, the method comprises configuring the material as an opioid pill or tablet comprising a bar code on at least one surface. In other examples, the method comprises configuring the material as a narcotic pill or tablet comprising a bar code on at least one surface. In some embodiments, the method comprises configuring the material as food comprising a bar code on at least one surface. In other instances, the method comprises configuring the material with an edible/dissolvable film or tape (which can be added to the material prior to ingestion) comprising the bar code. In some embodiments, the method comprises configuring the removable oral device to not alter a position of the user's teeth. In some embodiments, the method comprises configuring the removable oral device to not retain a position of the user's teeth. If desired, a removable oral device with a bar code reader may comprise a variable hardness across a palatal surface of the device.

In some embodiments, a method comprises updating an electronic medical record of a user by providing the electronic medical record of the user from a memory unit within a removable oral device, and providing new medical information to the electronic medical record in the memory unit to update the electronic medical record. In some instances, the removable oral device comprises a palatal element coupled to a clasping element with the palatal element configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The memory unit can be present within the body of the palatal element or on or in the clasping element if desired.

In certain embodiments, the method comprises wirelessly providing the new medical information to the electronic medical record in the memory unit to update the electronic medical record. In other examples, the method comprises configuring the removable oral device to not alter a position of the user's teeth. In some examples, the method comprises configuring the removable oral device to not retain a position of the user's teeth. If desired, a removable oral device designed to read/update an electronic medical record may comprise a variable hardness across a palatal surface of the device.

In another configuration, a method of monitoring human health comprises measuring at least one physiological parameter of a user using a removable oral device inserted into a mouth of the user. In some instances, the removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both) comprises at least one electrode positioned within the body of the palatal element and exposed to fluid entering and exiting the mouth of the user. The at least one electrode can be configured to measure the physiological parameter from fluid entering and exiting the mouth of the user.

In certain examples, the method comprises configuring the electrode to measure electrolyte levels in the fluid. In other examples, the method comprises configuring the electrode to measure ketone bodies in the fluid. In further examples, the method comprises configuring the electrode to measure salivary insulin levels in the fluid. In some embodiments, the method comprises configuring the electrode to measure a gas other than oxygen in the fluid. In other examples, the method comprises configuring the electrode to measure carbohydrate levels in the fluid. In some instances, the method comprises configuring the electrode to measure oxygen levels in the fluid. In certain embodiments, the method comprises configuring the electrode to measure carbon dioxide levels in the fluid. In other examples, the method comprises configuring the electrode to measure a respiration rate. In some embodiments, the method comprises configuring the electrode to measure fluid intake. In some embodiments, the method comprises configuring the electrode to measure heart rate. If desired, the method may comprise configuring the removable oral device with a second electrode configured to measure a different physiological parameter than the electrode. Suitable electrodes which can be used to measure these parameters include ion selective electrodes, material selective electrodes, electrodes designed to implement cyclic voltammetry or other electrochemical methods such as redox reactions designed to measure electrochemical potentials as a measure of the various fluid species. If desired, one or more reference electrodes may also be present and use with the electrode. Further, an on-board power source can be present to provide a voltage or current to the electrode(s). In some examples, the method comprises configuring the removable oral device to not alter a position of the user's teeth. In other examples, the method comprises configuring the removable oral device to not retain a position of the user's teeth. If desired, a removable oral device designed to monitor human health may comprise a variable hardness across a palatal surface of the device.

In another configuration, a method comprising monitoring head tremor comprising monitoring movement of the head using a removable oral device inserted into the mouth of the user is provided. The removable oral device comprises a palatal element coupled to a clasping element. The palatal element is configured to contact a roof of a user's mouth at a palatal surface, wherein the palatal element comprises a body comprising a thickness configured to lower a vaulted area of the roof of the mouth to decrease an overall oral volume of the mouth to a first oral volume, and wherein. The clasping element, when present, is configured to assist in removal of the removable oral device from the mouth. The palatal element (or the clasping element or both) comprises an accelerometer within the body of the palatal element, the accelerometer configured to detect head movement to monitor the head tremor, e.g., can detect head tremor by measuring movement of the removable oral device from a first position to a second position over a selected period.

In certain examples, the method comprises configuring the accelerometer to measure head movement at selected intervals. In other examples, the method comprises automatically switching the accelerometer off between the selected intervals where the accelerometer measures head movement. In some embodiments, the method comprises automatically switching the accelerometer on when the accelerometer measures head movement, e.g., switching the accelerometer on in response to a sensed temperature change after the user inserts the device into the mouth. In some embodiments, the method comprises configuring the removable oral device with a second accelerometer. In certain examples, the method comprises configuring the removable oral device to not alter a position of the user's teeth. In some embodiments, the method comprises configuring the removable oral device to not retain a position of the user's teeth. If desired, a removable oral device designed to monitor head tremor may comprise a variable hardness across a palatal surface of the device.

In another configuration, a method comprises monitoring head impact comprising monitoring movement of the head using a removable oral device inserted into the mouth of the user. In some instances, the removable oral device comprises a palatal element coupled to a clasping element, wherein the palatal element is configured to contact a roof of a user's mouth at a palatal surface, and wherein the palatal element comprises a body comprising a thickness configured to lower a vaulted area of the roof of the mouth to decrease an overall oral volume of the mouth to a first oral volume. The clasping element, when present, is configured to assist in removal of the removable oral device from the mouth. The palatal element (or clasping element or both) comprises an accelerometer within the body of the palatal element with the accelerometer configured to detect head movement to monitor the head impact. As compared to head tremor, head impact generally involves larger movements from a first position to a second position and/or the rate of movement is substantially faster.

In certain examples, the method comprises configuring the accelerometer to measure head impact at selected intervals. In other examples, the method comprises automatically switching the accelerometer off between the selected intervals where the accelerometer measures head impact. In some embodiments, the method comprises automatically switching the accelerometer on when the accelerometer measures head impact. In certain instances, the method comprises configuring the removable oral device with a second accelerometer to measure head impact. In some examples, the method comprises configuring the removable oral device with a motion sensor, wherein the accelerometer is automatically switched on when the motion sensor senses user motion. In certain embodiments, the method comprises configuring the removable oral device to not alter a position of the user's teeth. In some examples, the method comprises configuring the removable oral device to not retain a position of the user's teeth. If desired, a removable oral device designed to monitor head impact may comprise a variable hardness across a palatal surface of the device.

In certain embodiments, the methods described herein can be implemented or used with one or more processors, sensors, etc. described herein in connection with the removable oral device can be present or used with a computer system. In some instances, one or more of these components may be present on a storage case, a mobile device or both. The computer system/mobile device typically is separate from the removable oral device, but a processor, memory chip or other device may be integrated into some component of the removable oral device as desired. At least one processor can be electrically coupled to one or more memory units to receive input data and/or store any data. The processor may be, for example an Intel PENTIUM-type processor, Motorola PowerPC, Sun UltraSPARC, Hewlett-Packard PA-RISC processors, or any other type of processor. Various aspects of methods which use a removable oral device with an on-board processor may be implemented as specialized software. A processor can be, if desired, connected to one or more memory devices, such as a disk drive, memory, or other device for storing data. Memory is typically used for storing programs and data during use of the removable oral device. Electrical components of the oral device may be coupled by an interconnection device, which may include one or more buses (e.g., between components that are integrated within a same component) and/or a network (e.g., between components that reside on separate discrete components). The interconnection device provides for communications (e.g., signals, data, instructions) to be exchanged between components of the oral device. The processor and other components are electrically coupled to a power source. In addition, the removable oral device may comprise one or more interfaces that connect the processor to a separate device or system such as, for example, a communication network (in addition or as an alternative to the interconnection device). Illustrative interfaces include, but are not limited to, a serial ATA interface, ISA interface, PCI interface or the like or one or more wireless interfaces, e.g., a Bluetooth device, a WiFi device, a Near Field Communication device, a cellular device or other wireless protocols and/or interfaces.

In certain embodiments, the storage system of the removable oral device (or storage case) typically includes a readable and writeable nonvolatile recording medium in which data can be stored. The medium may, for example, be a solid state memory chip, solid state drive or flash memory. Typically, in operation, the processor causes data to be read from the nonvolatile recording medium into another memory that allows for faster access to the information by the processor than does the medium. This memory is typically a volatile, random access memory such as a dynamic random access memory (DRAM) or static memory (SRAM). It may be located in the storage system or in the memory system. The processor generally manipulates the data within the integrated circuit memory and then copies the data to the medium after processing is completed. A variety of mechanisms are known for managing data movement between the medium and the integrated circuit memory element and the technology is not limited thereto. The technology is also not limited to a particular memory system or storage system. In certain embodiments, the removable oral device may also include specially-programmed, special-purpose hardware, for example, an application-specific integrated circuit (ASIC) or a field programmable gate array (FPGA). Aspects of the technology may be implemented in software, hardware or firmware, or any combination thereof. The removable oral device may use or include a high-level computer programming language or specially programmed, special purpose hardware.

In certain examples, the processor and any associated sensors or components of the removable oral device may together define a computer platform for which application programs in high-level programming languages may be written. It should be understood that the technology is not limited to a particular computer system platform, processor, operating system, or network. Also, it should be apparent to those skilled in the art, given the benefit of this disclosure, that the present technology is not limited to a specific programming language or computer system. Further, it should be appreciated that other appropriate programming languages and other appropriate electrical components could also be used. In certain examples, the hardware or software can be configured to implement cognitive architecture, neural networks or other suitable implementations. In some instances, various configurations may be programmed using an object-oriented programming language, such as SmallTalk, Basic, Java, C++, Ada, or C# (C-Sharp). Other object-oriented programming languages may also be used. Alternatively, functional, scripting, and/or logical programming languages may be used. Various configurations may be implemented in a non-programmed environment (e.g., documents created in HTML, XML or other format that, when viewed in a window of a browser program, render aspects of a graphical-user interface (GUI) or perform other functions). Certain configurations may be implemented as programmed or non-programmed elements, or any combination thereof.

In certain embodiments, the methods of using the removable oral device may comprise (or interact with) a mobile device, e.g., a laptop, phone, tablet, etc. that is configured to control use of (or monitor usage of) the removable oral device. The mobile device may wirelessly communicate with the removable oral device to send signals and receive signals or data from the removable oral device. In addition, the mobile device can be pre-programmed or pre-configured to implement certain operations that can automatically load from the mobile device into the removable oral device. If desired, the mobile device can be designed for use with two or more different removable oral device to permit a single mobile device to implement the same or different operations on the two or more removable oral devices. The mobile device can couple to the removable oral device in a wired or wireless manner, e.g., using near field communication, Bluetooth, or other wireless devices and protocols, to send and receive information from the mobile device to the removable oral device. One or more menus can be present on the mobile device, e.g., in application software, to permit the user to select the particular methodology of using the removable oral device.

In certain embodiments, any one or more of the methods described herein can be used in combination with an app or application present on the mobile device. The application may permit remote control of the removable oral device and/or can be used to retrieve and/or store information such as usage values on the removable oral device.

In some instances, a method may comprise identifying a subject, e.g., a human, with a body mass index between 25-35 or between 27-35 or even more than 35 and then using the removable oral device to reduce their body mass index, e.g., to a level below 25. In some examples, the removable oral device can be used to reduce the BMI of the human subject to below 30, 29, 28, 27, 26 or 25. In other instances, the removable oral device can be used to reduce the BMI from an initial value to 5% less, 10% less, or even 20% less than the initial BMI value. In certain examples, the removable oral device can be used to reduce body fat percentage in with human males comprising a body fat percentage between 22-29% or exceeding 22% or 26% or with human females comprising a body fat percentage of 31-39% or exceeding 31%. In some examples, the removable oral device can be used, for example, until the subjects body fat percentage drops below a selected level, e.g., below a body fat percentage of 22% for human males and 31% for human females. For example, weight management can be attained by using the removable oral device and the frequency of use of the removable oral device can be changed once the desired weight, BMI or body fat percentage is reached.

In certain examples, the removable oral devices can be used in combination with a timer or timing circuit to monitor the duration of eating periods. The timer may automatically switch on when the device is inserted into the oral cavity and can switch off when the device is removed. The duration of eating can be transferred to the software application as desired. If the user is eating too quickly using the removable oral device, then a second removable oral device that decreases the oral volume even more could be used. Alternatively, the time it takes to consume food can be transferred to application software and used by a coach to provide guidance regarding mindful eating practices.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. A method of managing body weight in a human, the method comprising:
providing a removable oral device comprising an integral usage sensor, wherein the removable oral device comprises a palatal element coupled to a clasping element, wherein the palatal element is configured to contact a roof of a user's mouth at a palatal surface, wherein the palatal element comprises a body comprising a thickness configured to lower a vaulted area of the roof of the mouth to decrease an overall oral volume of the mouth during eating of food to a first oral volume to reduce consumption of amounts of the food, wherein the clasping element is configured to assist in removal of the removable oral device from the user's mouth, and wherein the removable oral device is configured to be inserted and retained in the mouth during the eating of the food, and wherein the removable oral device is configured not to alter or retain a position of the teeth with continued use of the removable oral device during eating of the food;
monitoring usage frequency of the removable oral device by transferring usage values from the usage sensor to an external device separate from the removable oral device; and
providing feedback to the human based on the transferred usage values to manage body weight in the human.

2. The method of claim 1, further comprising transferring the usage values from the usage sensor to a removable oral device storage case sized and arranged to receive and store the removable oral device.

3. The method of claim 1, further comprising transferring the usage values from the stored removable oral device to the storage case by providing an optical transmission from the stored removable oral device to the storage case.

4. The method of claim 3, further comprising configuring the optical transmission to comprise non-visible light wavelengths.

5. The method of claim 4, further comprising transferring the usage values received by the storage case to the external device.

6. The method of claim 4, further comprising transferring the usage values received by the storage case to a mobile device.

7. The method of claim 4, further comprising transferring the usage values received by the storage case to a wearable device.

8. The method of claim 1, further comprising transferring the usage values to a mobile device.

9. The method of claim 1, further comprising transferring the usage values to a wearable device.

10. The method of claim 1, wherein the providing feedback to the human based on the transferred usage values to manage body weight in the human comprises using a mobile device.

11. The method of claim 1, wherein the providing feedback to the human based on the transferred usage values to manage body weight in the human comprises using a wearable device.

12. The method of claim 1, wherein the providing feedback to the human based on the transferred usage values to manage body weight in the human comprises using an in-person coach.

13. The method of claim 1, wherein the removable oral device comprises a variable hardness with a hardness at an apex surface being greater than a hardness at edges of the removable oral device adjacent to teeth of the human when the removable oral device is inserted into the oral cavity of the human.

14. The method of claim 13, further comprising configuring an apex surface of the removable oral device with a first material comprising a Vickers hardness of at least 10 HV and configuring edges of the removable oral device to comprise a second material with a Vickers hardness of less than 10 HV.

15. The method of claim 13, further comprising configuring the usage sensor to comprise a temperature sensor, transferring the usage values from the usage sensor to a removable oral device storage case using a non-visible light transmission from the removable oral device to the removable oral device storage case, and wirelessly transferring the usage values received by the removable oral device storage case to a mobile device.

16. The method of claim 1, further comprising configuring the clasping element to comprise a wire comprising a coating on at least some portion of the wire.

17. The method of claim 1, further comprising configuring the usage sensor to comprise one or more of an optical sensor, a Bluetooth device, a temperature sensor, a cellular chip, a radio frequency transmitter, a processor, a memory unit, a transmitter or a receiver.

18-30. (canceled)
